(19) United States
(12) Patent Application Publication
(10) Pub. No.: US 2020/0018735 A1
(43) Pub. Date: Jan. 16, 2020

(54) RESPIRATION GAS MONITOR WITH AUTOMATED AND NONOBTRUSIVE FILTER CALIBRATION

(71) Applicant: KONINKLIJKE PHILIPS N.V., EINDHOVEN (NL)

(72) Inventor: Eugene Peter Gerety, Seymour, CT (US)

(21) Appl. No.: 16/495,583

(22) PCT Filed: Mar. 20, 2018
(86) PCT No.: PCT/EP2018/057077

§ 371 Date: Sep. 19, 2019

Related U.S. Application Data
(60) Provisional application No. 62/473,514, filed on Mar. 20, 2017.

(51) Int. Cl. G01N 33/00; A61B 5/08; A61B 5/097; G01N 21/3518

(52) U.S. Cl. CPC G01N 33/0006; A61B 5/082; A61B 5/0833; G01N 21/3518; A61B 5/097

(57) ABSTRACT

A respiration gas monitor (RGM) device includes an infrared light source (30) launching infrared light (34) through a respired air flow path (36), and an optical detector (40) that detects the infrared light after passing through the respired air flow path. An absorption line bandpass filter (42) has a passband encompassing an absorption line of a target gas. A reference line bandpass filter (44) has a passband over which the respired air is transparent. A control device (62, 64, 70, 80, 90) switches the RGM device between: a monitoring state in which the absorption line bandpass filter is in the path of the infrared light; and a calibration state in which the reference line bandpass filter is in the path of the infrared light and the absorption line bandpass filter is not in the path of the infrared light.

### FIELD

The following relates generally to the respiration gas monitor (RGM) device arts, gas detection cell calibration arts, and related arts.

### BACKGROUND

Respiration Gas Monitor (RGM) devices are used for measuring partial pressure or concentration of carbon dioxide (CO₂) in respired air, or some other respired gas such as oxygen (O₂), nitrous oxide (N₂O), or an administered anesthetic gas. An RGM device for measuring CO₂ is commonly referred to as a capnometer. Various gas component detection technologies may be employed. In certain RGM devices employing an optical detector, an infrared light source launches broadband infrared light that passes through a sampling cell through which respired air flows. The opposing optical detector module includes a narrowband filter and an infrared detector. The filter is tuned to pass a wavelength that is strongly absorbed by the target gas, e.g. 4.3 micron for CO₂.

In such a design, the optical detector is calibrated by measuring a reference signal in the absence of the target gas. This entails diverting the respired air flow from the sampling cell, and introducing flow of a reference gas such as air or nitrogen through the sampling cell. The reference gas is chosen to have negligible concentration of the target gas (e.g. negligible CO₂ in the case of a capnometer). Consequently, the measured signal output by the optical detector with the reference gas flowing is the maximum value, as there is negligible absorption by the target gas. With this reference signal determined, the ratio of the signal measured with the infrared light launched through the respired air flow versus the reference signal measured with the infrared light launched through the reference gas flow provides the signal reduction due to infrared absorption by the target gas in the respired air flow.

The following discloses new and improved systems and methods.

### SUMMARY

In one disclosed aspect, a respiration gas monitor (RGM) device comprises a respired air flow path for carrying respired air, an infrared light source arranged to launch infrared light through the respired air flow path, and an optical detector arranged to detect the infrared light after passing through the respired air flow path. An absorption line bandpass filter has a passband that encompasses an absorption line of a target gas. A reference line bandpass filter has a passband over which the respired air is transparent. A control device is operative to switch the RGM device between: a monitoring state in which the absorption line bandpass filter is in the path of the infrared light and the reference line bandpass filter is not in the path of the infrared light; and a calibration state in which the reference line bandpass filter is in the path of the infrared light and the absorption line bandpass filter is not in the path of the infrared light.

In another disclosed aspect, a method of operating a respiration gas monitor (RGM) device is disclosed. Respired air is flowed through a respired air flow path. Infrared light is launched through the respired air flow path. While flowing the respired air through the respired air flow path, target gas monitoring is performed, including measuring an infrared transmission signal indicating transmission of the launched infrared light through the respired air flow path with an absorption line bandpass filter disposed in the path of the infrared light and determining a value for the target gas in the respired air from the infrared transmission signal and a reference infrared signal. While flowing the respired air through the respired air flow path, a calibration is performed including measuring the reference infrared signal indicating transmission of the launched infrared light through the respired air flow path with a reference line bandpass filter disposed in the path of the infrared light.

In another disclosed aspect, a respiration gas monitor (RGM) device comprises: a respired air flow path for carrying respired air; a measurement device configured to measure optical transmission through the respired air flow path; a switched optical filtering device configured to switch between filtering the measured optical transmission using an absorption line bandpass filter and filtering the measured optical transmission using a reference line bandpass filter; and electronics configured to output a concentration or partial pressure of the target gas in the respired air using a comparison of the measured optical transmission filtered using the absorption line bandpass filter and the measured optical transmission filtered using the reference line bandpass filter.

One advantage resides in providing more accurate monitoring of carbon dioxide or another target gas in respired air.

Another advantage resides in providing more frequent calibration of a respiration gas monitor (RGM) device.

Another advantage resides in providing an RGM device that does not require connection to nitrogen or another calibration gas.

Another advantage resides in providing an RGM device in which the respired gas flow through the RGM device is not interrupted to perform calibration.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a respiration gas monitor (RGM) device.

FIGS. 2-7 diagrammatically illustrate embodiments of a control device of the RGM device of FIG. 1 operative to switch the RGM device between a monitoring state, in which the target gas is monitored, and a calibration state.

FIG. 8 diagrammatically shows an operational flow chart for operation of the RGM device of FIG. 1.

### DETAILED DESCRIPTION

In a respiration gas monitor (RGM) device employing optical detection of the target gas, the infrared light source typically includes an infrared emitting element (e.g. a ceramic element) that is resistively heated by conducting an electrical current pulse train through the infrared element. The infrared element is resistively heated is to a temperature effective to produce broadband blackbody radiation with strong emission in the infrared. This infrared emission is strongly dependent on the precise temperature of the heated infrared element. The temperature of the heated infrared emitting element can drift over time during operation of the RGM device. The infrared light detector may be a lead selenide (PbSe) detector, a microbolometer, thermocouple, pyroelectric detector, or the like, and its sensitivity is also usually strongly temperature dependent. The detector temperature can also drift over time during operation of the RGM device. Depending upon the thermal stabilities of the IR source and sensor, the calibration of the optical detection cell may need to be repeated at intervals as frequent as every few minutes to tens of minutes to ensure sufficient accuracy for robust medical monitoring of a critically ill patient.

In existing RGM devices, the calibration of the optical detection cell is performed by flow switching, in which the respired air flow is diverted away from the sampling cell and the reference gas flow (e.g. nitrogen gas or air) is flowed through the sampling cell. This approach has certain disadvantages as recognized herein. In the case of a reference gas other than air, such as nitrogen, the reference gas must be available in the patient's hospital room and connected with the RGM device. If air is used as the reference gas, then there is potential for error due to residual levels of the target gas (e.g. CO₂ in the case of a capnometer) which may be present in the air.

Another disadvantage recognized herein is that the respiration gas monitoring is interrupted for a time interval as the flow through the sampling chamber of the RGM device is switched from the respiration gas flow to the reference gas flow, and then is switched back from the reference gas flow to the respiration gas flow. Besides the apparent interruption of monitoring data, there is the potential for generating erroneous measurements if the respiratory gas monitoring measurements are resumed before the respiration gas flow through the sampling cell is reinstated and reaches equilibrium.

In view of these difficulties, it is disclosed herein to provide a respiration gas monitor (RGM) device that includes an infrared light source launching infrared light through a respired air flow path, and an optical detector that detects the infrared light after passing through the respired air flow path. An absorption line bandpass filter has a passband encompassing an absorption line of a target gas. A reference line bandpass filter has a passband over which the respired air is transparent. A control device switches the RGM device between: a monitoring state in which the absorption line bandpass filter is in the path of the infrared light; and a calibration state in which the reference line bandpass filter is in the path of the infrared light and the absorption line bandpass filter is not in the path of the infrared light. In this way, the calibration can be performed rapidly, without requiring any interruption of the flow of the respired air through the respired air flow path.

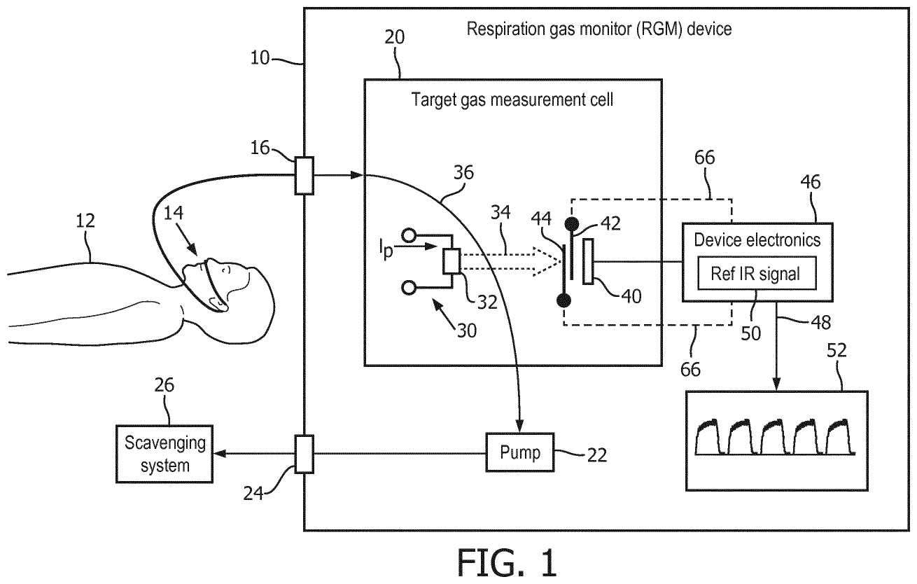

With reference to FIG. 1, an illustrative respiration gas monitor (RGM) device 10 is connected with a patient 12 by a suitable patient accessory, such as a nasal cannula 14 in the illustrative example, or by an airway adaptor connecting with an endotracheal tube used for mechanical ventilation, or so forth. The patient accessory 14 may optionally include one or more ancillary components, such as an air filter, water trap, or the like (not shown). In the illustrative RGM device 10, respired air is drawn from the patient accessory 14 into an air inlet 16 and through target gas measurement cell 20 by a pump 22. The respired air is then discharged via an air outlet 24 of the RGM device 10 to atmosphere or, as in the illustrative embodiment, is discharged through the air outlet 24 into a scavenging system 26 to remove an inhaled anesthetic or other inhaled medicinal agent before discharge into the atmosphere. It should be noted that the respired air generally has a composition that is different from the ambient air, for example the respired air may contain different concentrations of CO₂, oxygen, and/or may contain added gas such as an administered anesthetic gas.

The illustrative RGM device setup has a sidestream configuration in which respired air is drawn into the RGM device using the pump 22, and the target gas measurement cell 20 is located inside the RGM device 10. The sidestream configuration is suitably used for a spontaneously breathing patient, i.e. a patient who is breathing on his or her own without assistance of a mechanical ventilator. In an alternative configuration, known as a mainstream configuration (not illustrated), the target gas measurement cell is located externally from the RGM device housing, typically as a target gas measurement cell patient accessory that is inserted into the “mainstream” airway flow of the patient. Such a mainstream configuration may, for example, be employed in conjunction with a mechanically ventilated patient, with the target gas measurement cell patient accessory being designed to mate into an accessory receptacle of the ventilator unit, or is installed on an airway hose feeding into the ventilator.

The target gas measurement cell 20 comprises an infrared optical absorption cell in which the target gas in the respired air drawn from the patient accessory 14 produces absorption in the infrared that is detected optically. By way of non-limiting illustration, CO₂ has an absorption peak at about 4.3 micron. By way of further non-limiting illustration, other target gases may include oxygen (O₂), nitrous oxide (N₂O), or an administered anesthetic gas, each of which have specific characteristic absorption lines in the infrared. An infrared light source 30 includes an infrared emitting element 32 that is resistively heated by a drive current I_{P} which in some embodiments comprises an electric current pulse train. The electric current I_{P} heats the infrared emitting element (e.g. a ceramic element or other thermally radiating element) to emit blackbody radiation with strong emission in the infrared. Thusly launched broadband infrared light 34 (diagrammatically indicated by a block arrow in FIG. 1) transmits through a respired air flow path 36 (diagrammatically indicated by a curve arrow in FIG. 1) along which the respired air flows. The flow path 36 may be defined by a tube or other conduit defining a cuvette with walls made of a plastic, glass, sapphire, or other material that is substantially transparent for the infrared light 34. The pump 22 actively drives the flow of respired air through the flow path 36; in a mainstream configuration the flow may be driven by mechanical ventilation of the patient, and/or by active breathing of the patient.

An optical detector 40 is configured to detect the infrared light 34. In some illustrative embodiments, the optical detector 40 may be a lead selenide (PbSe) detector, a microbolometer, thermocouple, pyroelectric detector, or the like. To provide specificity to the target gas, an absorption line bandpass filter 42 having a passband tuned to an absorption line of the target gas is interposed between the infrared emitting element 32 and the optical detector 40. For example, in the case of the target gas being CO₂, the absorption line bandpass filter 42 suitably has a passband that encompasses, and is preferably centered at, 4.3 micron which is a wavelength at which carbon dioxide is strongly absorbing. For other target gases, the bandpass filter is designed to have a passband that encompasses, and preferably is centered on, a strong absorption line of the other target gas. The absorption line bandpass filter may, for example, comprise a stack of layers on an infrared light-transmissive substrate such as sapphire, in which the layers of the stack of layers have thicknesses, refractive indices, and arrangement designed to form an interference filter with a narrow passband having the requisite center frequency (e.g. 4.3 micron for CO₂ detection).

For performing the calibration, the illustrative embodiment of FIG. 1 employs a separate reference line bandpass filter 44 constructed similarly to the absorption line bandpass filter 42, but with the stack of layers tuned to a wavelength at which no component present in non-negligible quantities in the respired air exhibits strong absorption. In some embodiments, the reference line bandpass filter 44 has a narrow passband that encompasses, and preferably is centered on, a frequency of 3.6 micron, although other wavelengths for which the respired air is transparent are also contemplated. In general, the reference line bandpass filter 44 has a passband over which the respired air carried in the respired air flow path 36 is transparent. By “transparent” it is meant that absorption by the respired air is negligibly small over the passband of the reference line bandpass filter 44 when compared with absorption by the target gas over the passband of the absorption line bandpass filter 42 for the lowest concentration or partial pressure of the target gas in the respired air that the RGM device 10 is designed to detect.

The RGM device 10 further includes RGM device electronics 46 that provide electrical biasing of, and readout for, the optical detector 40. The electronics 46 optionally provide the drive current I_{P} for the infrared light source 30 (connection not shown in FIG. 1; moreover other driving configurations are contemplated such as a separate current or voltage drive power supply). The RGM device electronics 46 also include analog signal processing circuitry and/or digital signal processing (DSP) suitable for converting the detected signal into a gas signal 48, e.g. a concentration or partial pressure of CO₂ in the respired air flow, and optionally for performing further processing such as detecting a breath interval and/or an end-tidal CO₂ level (etCO₂ level, relevant for capnometry embodiments). The conversion to CO₂ level or other target gas signal can employ suitable empirical calibration—for example, in general, concentration or partial pressure of the target gas produces greater absorption and hence a reduced signal from the optical detector 40. The empirical calibration may take into account other factors such as flow rate or pressure, and/or the effects of gases other than the target gas (for example, oxygen and nitrous oxide are known to affect the infrared absorption characteristics due to CO₂), and can be suitably programmed as a look-up table, mathematical equation, non-linear op-amp circuit, or so forth.

Another aspect of the conversion is compensating for drift in the signal output by the optical detector 40. Such drift may be due to drift in the detector 40, and/or due to drift in the intensity of the infrared radiation 34 launched by the infrared light source 30, and/or due to other factors such as condensate buildup on walls of the path 36 through which the respired air flows. To account for such drift, a calibration is occasionally performed using the reference line bandpass filter 44 as described elsewhere herein in order to generate a reference infrared (IR) signal 50. By employing a ratio of the signal from the optical detector 40 versus the reference IR signal 50, such drift is compensated.

In the case of the RGM device electronics 46 being implemented at least in part by DSP, such DSP may be implemented by a microcontroller or microprocessor or the like programmed by instructions stored on a read only memory (ROM), electronically programmable read-only memory (EPROM), CMOS memory, flash memory, or other electronic, magnetic, optical or other non-transitory storage medium that is readable and executable by the microcontroller or microprocessor or the like to perform the digital signal processing. For DSP processing, a front-end analog-to-digital (A/D) conversion circuit is typically provided to digitize the detector signal from the optical detector 40. To provide useful target gas monitoring, an output component 52 is provided. In the illustrative embodiment, the output component 52 is a display 52, e.g. an LCD display or the like. The illustrative display 52 plots target gas concentration or partial pressure versus time as a trend line. Additionally or alternatively, the display may show a numerical value, e.g. of the target gas concentration at a particular time in the respiratory cycle, e.g. etCO₂ in the case of a capnometer. The output component may additionally or alternatively take other forms, such as being or including (possibly in addition to the display 52) a USB port or other data port via which the target gas data may be read out.

It will be further appreciated that the RGM device 10 may include numerous other components not illustrated in simplified diagrammatic FIG. 1, such as a pressure gauge and/or flow meter for monitoring the respired air flow, a keypad or other user input components, and/or so forth.

FIG. 1 illustrates both the absorption line bandpass filter 42 and the reference line bandpass filter 44 in the path of the infrared light 34. In actual operation, however, a control device is operative to switch the RGM device 10 between a monitoring state and a calibration state. In the monitoring state, the absorption line bandpass filter 42 is in the path of the infrared light 34 and the reference line bandpass filter 44 is not in the path of the infrared light 34. The monitoring state is the normal operating state, and provides for the optical detector 40 to measure the absorption of the infrared light due to the target gas (e.g. CO₂), since the absorption line bandpass filter 42 encompasses (and is preferably centered on) an absorption line of the target gas (e.g. 4.3 micron in the case of CO₂target gas). When switched to the calibration state, the reference line bandpass filter 44 is in the path of the infrared light 34 and the absorption line bandpass filter 42 is not in the path of the infrared light 34. This state allows for measurement of the reference infrared signal 50, since the reference line bandpass filter 44 is chosen to have a passband over which the respired air is transparent (e.g., 3.6 micron in illustrative examples herein). In this way, the reference infrared signal 50 can be measured without changing out the gas flow from respired gas to air or nitrogen. In the following, some illustrative embodiments of the control device for switching between the monitoring state and the calibration state are described.

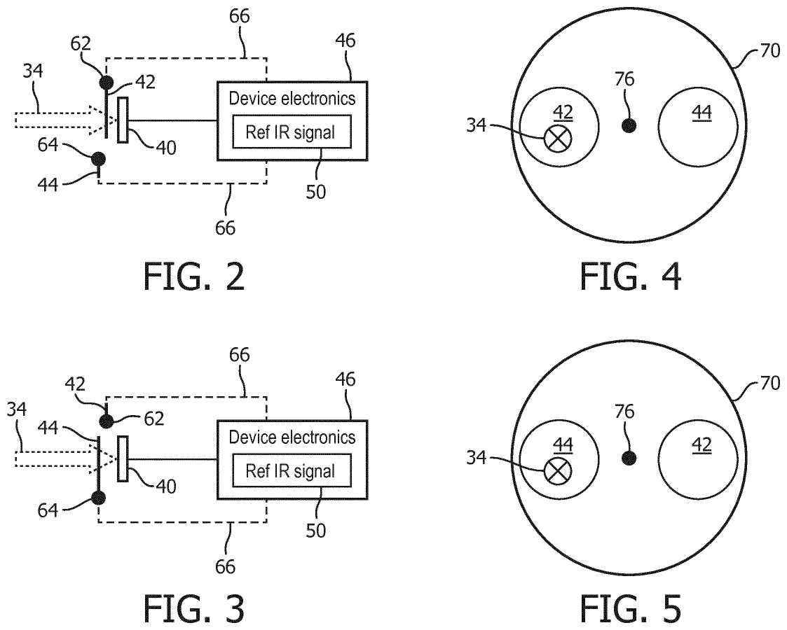

With reference to FIGS. 2 and 3, in one illustrative embodiment the control device comprises a filter flipper comprising a first filter flipper 62 that mechanically moves the absorption line bandpass filter 42 into or out of the path of the infrared light 34, and a second filter flipper 64 that mechanically moves the reference line bandpass filter 44 into or out of the path of the infrared light 34. The two filter flippers 62, 64 are coupled together, e.g. mechanically or by control logic or electrical control circuitry 66 (e.g., connected with or part of the device electronics 46 as diagrammatically indicated in FIGS. 1-3), so as to switch to the monitoring state by the first filter flipper 62 mechanically moving the absorption line bandpass filter 42 into the path of the infrared light 34 and the second filter flipper 64 mechanically moving the reference line bandpass filter 44 outside of the path of the infrared light 34, as shown in FIG. 2. The two filter flippers 62, 64 are similarly switchable to the calibration state by the second filter flipper 64 mechanically moving the reference line bandpass filter 44 into the path of the infrared light 34 and the first filter flipper 62 mechanically moving the absorption line bandpass filter 44 outside of the path of the infrared light. In some embodiments, the switching between the monitoring state and the calibration state is fast, e.g. 0.2 sec in some contemplated embodiments. In some embodiments the RGM 10 switches from the monitoring state (FIG. 2) to the calibration state (FIG. 3), measures the reference infrared signal 50, and switches back to the monitoring state (FIG. 2) sufficiently quickly compared with a single respiration cycle (e.g. 10-20 sec for a respiration rate of 3-6 breaths per minute) so that the sampling of the target gas waveform is advantageously not significantly disturbed by the calibration.

The design of the filter flipper assembly 62, 64 can take various forms. In one embodiment each bandpass filter 42, 44 is mounted on a rotating axis and the two filter flippers 62, 64 are motors that rotate the respective filters 42, 44 about those axes into and out of the path of the infrared light 34, as diagrammatically shown in FIGS. 2 and 3.

With reference to FIGS. 4 and 5, in another illustrative embodiment the filter flipper comprises a filter wheel 70 that rotates about an axis 76, and on which both the absorption line bandpass filter 42 and the reference line bandpass filter 44 are mounted. FIGS. 4 and 5 illustrate the monitoring state (FIG. 4) and the calibration state (FIG. 5), respectively, viewed along the optical axis of the infrared light 34. In the monitoring state shown in FIG. 4, the filter wheel 70 is rotated about its axis 76 such that the absorption line bandpass filter 42 is rotated into the infrared light 34. In the calibration state shown in FIG. 5, the filter wheel 70 is rotated about its axis 76 such that the reference line bandpass filter 44 is rotated into the infrared light 34. In this design the control device is connected to rotate the filter wheel 70 to implement switching between the monitoring and calibration states.

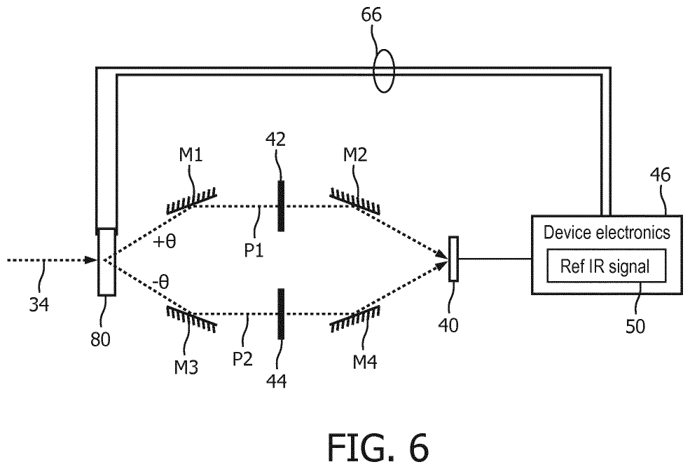

With reference to FIG. 6, in another illustrative embodiment, the control device comprises an electro-optical beam steering device 80 operable at a first electric bias or at a second electric bias provided by the electrical control circuitry 66 driven by the device electronics 46. The optical beam steering device 80 may, for example, employ a liquid crystal device forming a switchable diffraction grating. Such electro-optical beam-steering devices are used, for example, in bar-code scanners to provide fast beam direction switching. When biased at the first electric bias, the illustrative electro-optical beam steering device 80 steers the infrared light 34 at an angle +θ to be reflected by mirrors M1, M2 along a first optical path P1 that passes through the absorption line bandpass filter 42 (and does not pass through the reference line bandpass filter 44) and then impinges upon the optical detector 40. When biased at the second electric bias, the illustrative electro-optical beam steering device 80 steers the infrared light 34 at an angle −θ to be reflected by mirrors M3, M4 along a second optical path P2 that passes through the reference line bandpass filter 44 (and does not pass through the absorption line bandpass filter 42) and then impinges upon the optical detector 40.

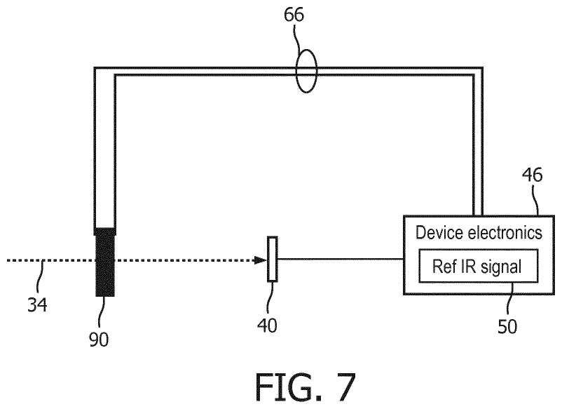

With reference to FIG. 7, in another illustrative embodiment, the control device comprises an electrically tunable optical bandpass filter 90 operable at a first electric bias or a second electric bias. The electrically tunable optical bandpass filter 90 may, for example, comprise a liquid crystal or acousto-optic device comprising an electrically tunable interference filter that can be switched between two different passbands, e.g. 4.3 micron in the illustrative CO₂ target gas example, and 3.6 micron as the reference passband. The electrically tunable optical bandpass filter 90 is always disposed in the path of the infrared light 34, but can be tuned to instantiate either the absorption line bandpass filter 42 or the reference line bandpass filter 44. More particularly, when biased at the first electric bias, the monitoring state is implemented by tuning the electrically tunable optical bandpass filter 90 to the passband of the absorption line bandpass filter 42, whereby the electrically tunable optical bandpass filter 90 instantiates the absorption line bandpass filter 42. When biased at the second electric bias, the calibration state is implemented by tuning the electrically tunable optical bandpass filter 90 to the passband of the reference line bandpass filter 44, whereby the electrically tunable optical bandpass filter 90 instantiates the reference line bandpass filter 44.

The control device embodiments of FIGS. 2-7 are merely illustrative examples, and other configurations are contemplated. Advantageously, the control device 62, 64, 70, 80, 90 is not operative to divert flow of respired air through the respired air flow path 36 when operating to switch the RGM device 10 to the calibration state. Rather, the control device switches between the monitoring state and the calibration state, and indeed performs the calibration, without the need to interrupt or alter flow of respired air through the respired air flow path 36. This promotes stability of the respired air flow and eliminates transient periods when the respired air flow may not yet be stabilized after a calibration operation, and thereby improves accuracy of the RGM device 10.

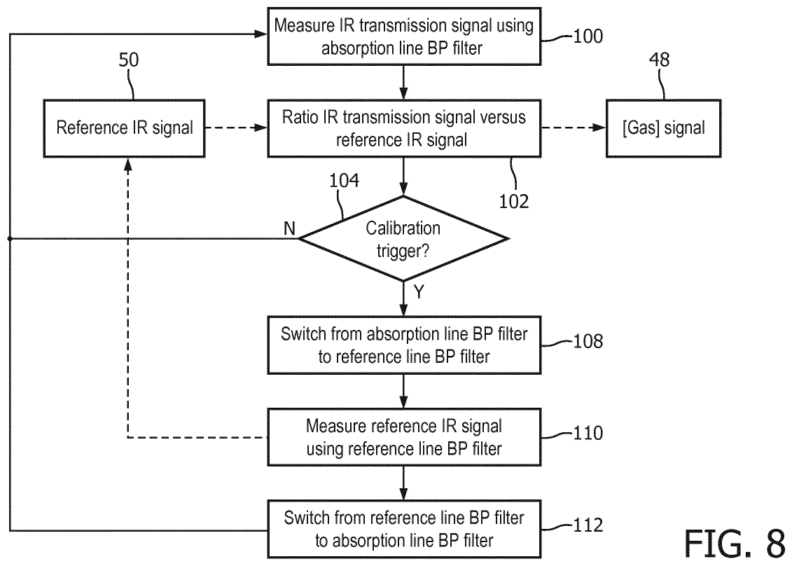

With reference to FIG. 8, operation of the RGM device 10 of FIG. 1 is diagrammatically flowcharted. The monitoring state is depicted by operation 100 in which the infrared (IR) transmission signal is measured using the absorption line bandpass (BP) filter 42 and an operation 102 in which a ratio of the IR transmission signal and the reference IR signal 50 is computed to generate the target gas concentration or partial pressure signal 48. As previously noted, the signal processing operation 102 may optionally account other factors such as flow rate or pressure, and/or the effects of gases other than the target gas (e.g. oxygen and/or nitrous oxide which can affect the infrared absorption characteristics due to CO₂), and these can be suitably programmed as a look-up table, mathematical equation, non-linear op- amp circuit, or so forth.

At a decision operation 104, it is determined whether the RGM device 10 should switch from the monitoring state to the calibration state in order to update the value of the reference IR signal 50. The decision 104 can be based on various chosen factors. In one embodiment, the decision 104 switches to the calibration state after a fixed time interval, e.g. after every 5 minutes of monitoring. In another embodiment, the decision 104 switches when the target gas signal 48 drifts over time by more than some threshold amount, in order to ensure that the drift is not due to a change in the reference signal (e.g. due to drift of the intensity of the infrared light 34 output by the infrared light source 30, or due to drift of the optical detector 40, or due to contamination of the walls of the respired air flow path 36, or so forth). So long as the decision operation 104 does not call for updating the calibration, flow returns to the monitoring state IR transmission measurement operation 100 to continue monitoring the target gas.

On the other hand, if the decision operation 104 calls for a calibration update, then in an operation 108 the RGM device 10 switches from the monitoring state to the calibration state by switching from the absorption line bandpass filter 42 to the reference line bandpass filter 44, e.g. using any one of the control devices described with reference to FIGS. 2-7. Now in the calibration state, a measurement operation 110 is performed which is analogous to the measurement operation 100 except now with the infrared light being filtered by the reference bandpass filter. This measurement 110 then provides the updated value for the reference infrared signal 50. In an operation 112, the RGM device 10 switches from the calibration state back to the monitoring state by switching from the reference line bandpass filter 44 back to the absorption line bandpass filter 42. Flow then returns to operation 100 to resume monitoring of the target gas. Advantageously, the calibration process sequence 108, 110, 112 involves only a rapid change-out of the bandpass filters 42, 44, e.g. by a mechanical filter flipper (e.g. illustrative embodiments of FIGS. 2-5), fast electro-optical beam steering (e.g. illustrative embodiment of FIG. 6), or by using a tunable bandpass optical filter to instantiate the requisite filter (e.g. illustrative embodiment of FIG. 7). Thus, the calibration can be performed rapidly so as to introduce negligible interruption in the [CO₂] waveform or other target gas waveform generated by the monitoring state. This in turn facilitates more frequent updating of the reference infrared signal 50 and consequently more accurate monitoring of the target gas.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A respiration gas monitor (RGM) device comprising:
a respired air flow path for carrying respired air;
an infrared light source arranged to launch infrared light through the respired air flow path;
an optical detector arranged to detect the infrared light after passing through the respired air flow path;
an absorption line bandpass filter having a passband that encompasses an absorption line of a target gas;
a reference line bandpass filter having a passband over which the respired air is transparent; and
a control device operative to switch the RGM device between:
a monitoring state in which the absorption line bandpass filter is in the path of the infrared light and the reference line bandpass filter is not in the path of the infrared light, and
a calibration state in which the reference line bandpass filter is in the path of the infrared light and the absorption line bandpass filter is not in the path of the infrared light: wherein the control device comprises:
an electro-optical beam steering device operable at:
a first electric bias implementing the monitoring state by steering the infrared light to an optical path (P1) that passes through the absorption line bandpass filter and does not pass through the reference line bandpass filter, and
a second electric bias implementing the calibration state by steering the infrared light to an optical path (P2) that passes through the reference line bandpass filter and does not pass through absorption line bandpass filter:
wherein the control device comprises:
an electro-optical beam steering device operable at:
a first electric bias implementing the monitoring state by steering the infrared light to an optical path (P1) that passes through the absorption line bandpass filter and does not pass through the reference line bandpass filter, and
a second electric bias implementing the calibration state by steering the infrared light to an optical path (P2) that passes through the reference line bandpass filter and does not pass through the absorption line bandpass filter.

2-5. (canceled)

6. The RGM device of claim 1 wherein the reference line bandpass filter has a passband encompassing 3.6 micron.

7. The RGM device of claim 1 wherein the target gas is carbon dioxide and the absorption line bandpass filter has a passband encompassing the 4.3 micron absorption line of carbon dioxide.

8. The RGM device of claim 1 further comprising:
electronics configured to:
measure a reference infrared signal using the optical detector with the RGM device in the calibration state, and to
measure a concentration or partial pressure of the target gas in the respired air using the optical detector with the RGM device in the monitoring state and further using the reference infrared signal.

9. The RGM device of claim 1 wherein the control device is not operative to divert flow of respired air through the respired air flow path when operating to switch the RGM device to the calibration state.

10. A method of operating a respiration gas monitor (RGM) device, the method comprising:
flowing respired air through a respired air flow path;
launching infrared light through the respired air flow path;
while flowing the respired air through the respired air flow path, performing target gas monitoring including measuring an infrared transmission signal indicating transmission of the launched infrared light through the respired air flow path with an absorption line bandpass filter disposed in the path of the infrared light and determining a value for the target gas in the respired air from the infrared transmission signal and a reference infrared signal; and
while flowing the respired air through the respired air flow path, performing a calibration including measuring the reference infrared signal indicating transmission of the launched infrared light through the respired air flow path with a reference line bandpass filter disposed in the path of the infrared light:
wherein:
the target gas monitoring is performed with the RGM device in a monitoring state in which an electrically tunable optical bandpass filter instantiates the absorption line bandpass filter by being tuned to a passband that encompasses an absorption line of a target gas; and
the calibration is performed with the RGM device in a calibration state in which the electrically tunable optical bandpass filter instantiates the reference line bandpass filter by being tuned to a passband over which the respired air is transparent.

11. The method of claim 10 wherein:
the target gas monitoring is performed with the RGM device in a monitoring state in which the absorption line bandpass filter is disposed in the path of the infrared light and the reference line bandpass filter is disposed outside of the path of the infrared light, and
the calibration is performed with the RGM device in a calibration state in which the reference line bandpass filter is disposed in the path of the infrared light and the absorption line bandpass filter is disposed outside of the path of the infrared light.

12-14. (canceled)

15. The method of claim 10 wherein the absorption line bandpass filter has a passband that encompasses an absorption line of a target gas and the reference line bandpass filter has a passband over which the respired air is transparent.

16-20. (canceled)
